(19) United States
(12) Patent Application Publication
(10) Pub. No.: US 2023/0143155 A1
(43) Pub. Date: May 11, 2023

(54) Apparatus for Supporting a Person in a Prone Position Comprising a Movable Platform

(71) Applicant: Mirjana Prokic, London (GB)

(72) Inventors: Alistar Din Fe Magrini, London (GB); Jeremy Wright, London (GB)

(21) Appl. No.: 17/917,607

(22) PCT Filed: Mar. 25, 2021
(86) PCT No.: PCT/GB2021/050739

§ 371 Date: Oct. 7, 2022

(30) Foreign Application Priority Data
Apr. 9, 2020 (GB) 2005294.0

(51) Int. Cl. A61H 1/00; A47C 9/00

(52) U.S. Cl. CPC A61H 1/005; A47C 9/005; A61H 1/003; A61H 2201/1676

(57) ABSTRACT

Apparatus for supporting a person in a prone position comprises a support stand and a platform movably mounted to the support stand by a linkage, in which the platform is movable on the linkage between a first position and a second position, in which the platform comprises an upper portion in a first plane and a lower portion comprising a concavity relative to the first plane.

The present invention relates to apparatus for supporting a person in a prone position comprising a movable platform, for use particularly, but not exclusively, to position a user according to the Alexander Technique. In addition to this, the movability of the platform can provide a variety of different advantageous movements to induce relaxation or sleep and/or to facilitate ease of use.

Modern living can be stressful, and interaction with the modern world can cause issues with posture. For example, unsuitable or incorrect positions adopted when sitting at a desk at work, when driving or operating machinery, or when simply sitting for prolonged periods of time on poor quality seating, such as is commonly provided on public transport or at transport terminals, can all cause back and neck pain. Furthermore, stress caused by work pressure, social pressure, or general anxiety during travel or other activities can interfere with a person’s ability to relax or to sleep. The present invention is intended to address some of these issues by providing an apparatus which combines a particular postural methodology with a particular sleep induction technique.

The Alexander Technique, named after its creator Frederick Matthias Alexander, involves teaching users to adopt particular postures and movements in their daily lives. It mainly involves ways of standing, sitting or lying down, and moving efficiently between positions while maintaining a comfortable relationship between the head, neck and spine. One particular position taught by the Alexander Technique is a partial squat called monkey. This involves a person folding at the hips, knees and ankles while maintaining the the integrity of the head, neck and back relationship. It is a position commonly adopted by toddlers when reaching for the floor, but generally not by most adults. It is considered a beneficial poise to adopt because the back and neck are kept in a naturally straight position rather than being bent or flexed, while the hips, knees and ankles are bent into a naturally relaxed position. Done correctly the monkey position opens the back out, stimulates breathing and integrates the limbs with the core of the body. It is a position seen in some martial arts and sports where balance and power are sought, for example a lunge in fencing. It is now believed that simply placing a person in a relaxed prone position like monkey helps to alleviate stress and reduce back and neck pain. These are issues which many adults experience, in particular at times of increased stress such as when traveling.

It has long been known that rocking can induce sleep in infants, but recent studies into the matter have now demonstrated that the same effects can be achieved with adults. For example, the study “Rocking Synchronizes Brain Waves During a Short Nap”, by Bayer et al, published in Current Biology, Volume 21, Issue 12, 21 Jun. 2011, establishes that swinging can modulate physiological parameters of human sleep. A bed which rocked side to side at a frequency of 0.25 Hz facilitated the transition from waking to sleep in volunteers, and increased the duration of stage N2 sleep. It was found that the rocking motion also induced a sustained boosting of slow oscillations and spindle activity. It was concluded that sensory stimulation associated with a swinging motion exerts a synchronizing action in the brain that reinforces endogenous sleep rhythms. These conclusions were more recently supported by the study: “Rocking Promotes Sleep In Mice Through Rhythmic Stimulation Of The Vestibular System”, by Kompotis et al, published in Current Biology, Volume 29, Feb. 4, 2019. This study demonstrated that rocking promotes sleep in species other than humans, and provided the first evidence that the vestibular otolithic organs mediate sleep promotion and that this effect depends on the linear acceleration applied, and not to the rocking frequency. In this study the mice subjects were rocked back and forth along a horizontal plane.

Therefore, according to the present invention apparatus for supporting a person in a prone position comprises a support stand and a platform movably mounted to said support stand by a linkage, in which said platform is movable on said linkage between a first position and a second position, in which said platform comprises an upper portion in a first plane, and a lower portion comprising a concavity relative to said first plane.

Thus, the apparatus of the present invention provides a platform for supporting a user in a face-down prone position in which their back and neck can remain in a neutral aligned position on the upper portion, while their hips can assume a relaxed rotated pelvic position relative to their spine at the junction between the upper portion and the lower potion, and their knees can assume a relaxed bent position in the concavity of the lower portion. This is essentially the Alexander Technique monkey position described above. The linkage between the support stand and the platform can then provide a variety of different advantageous movements to induce relaxation or sleep and/or to facilitate ease of use.

Firstly, in one version of the invention the linkage can comprise a rotational linkage which can support the platform for rotation about an external axis displaced from the platform. The external axis can be aligned with a longitudinal extent of the platform, so it rocks from side to side, however preferably it can be aligned with a lateral extent of the platform, so it rocks back and forth. There are a number of different ways this can be done. For example, it can be done by pivoting the platform about a fixed point, so the whole platform simply swings back and forth along an arc like a normal swing. However, this may not be ideal because the extent of the angular change of the platform as it swings between the first and second positions may place too much pressure on different parts of the user’s body, which may not be particularly conducive to relaxation.

Alternatively, the linkage can be a parallel linkage in which two spaced internal axes of the platform are rotated about two corresponding fixed points. This can be done so the platform remains at a constant angle while travelling along an arc, like a glider swing. Therefore, in one version of the invention the rotational linkage can support the platform at an angle to the support stand in the first position, and then it can support the platform such that it remains at that angle between the first position and the second position.

In this configuration an internal axis of the platform which is equidistant between the two spaced internal axes referred to above is effectively that which defines the shape of the arc along which the platform travels, and which rotates about the external axis. This kind of arrangement would be adopted if it was required to generate a rocking motion without any rotation of the platform on its own axis. This might be the case if further research demonstrated that this was the most effective way to induce relaxation or sleep.

However, following on from the above, the rocking motion can be a hybrid of the two above described arrangements. As such, the rotational linkage can support the platform for independent rotation about an internal axis passing through the platform during rotation of the platform about the external axis. The rotation of the platform about the internal axis is “independent” of the rotation about the external axis in the sense that it is not related to the angular change the platform would normally experience during rotation about the external axis in its own. The support stand can comprise an underside in a second plane, upon which it stands in use. Therefore, if the apparatus is placed on a level surface in use, the second plane is effectively horizontal. The rotational linkage can then support the platform at a first oblique angle to the second plane in the first position, and at a second oblique angle to the second plane in the second position.

It is not yet fully known if this hybrid arrangement will be the most effective at inducing relaxation or sleep, but that is the inventors’ hypothesis. The movement experienced by the user with this configuration will be a specific chosen combination of degrees of longitudinal reciprocation (back and forth) of the platform relative to the support stand, medial reciprocation (up and down) of the platform relative to the support stand during the longitudinal reciprocation, these two movements combining as the platform rotates about the external axis (swinging), and transverse axial rotation (rotation) of the platform about the internal axis. The skilled person can apply and adjust these movements relative to one another to achieve different results. For example, they may prefer flatter or deeper swinging rotational movements with a greater or lesser ratio of longitudinal to medial reciprocation respectively, and/or they may prefer less or more rotation about the internal axis as this occurs.

In addition to the above, it will be a matter for the skilled person to determine the speed and acceleration of the reciprocal movements which occur. For example, in one version of the invention one full oscillation can take 4 seconds.

In terms of mechanically implementing any of the above described movements, this can be done in any known way that two linked parts can be made to move relative to one another. However, in preferred versions of the invention in which the rotational linkage provides a hybrid movement like that described above, the rotational linkage can comprise a first extension on a first side of the platform, which first extension can be located in a first guide provided in the support stand, and a second extension on the first side of the platform, which second extension can be located in a second guide provided in the support stand. The first extension can be located on a first lateral axis of the platform, and the second extension can be located on a second lateral axis of the platform which is parallel to the first lateral axis and is disposed between the first lateral axis and an upper end of the platform. The first guide can have a first curved shape, and the second guide can have a second curved shape different to the first curved shape. The first extension and the second extension can be simultaneously moveable through the first guide and the second guide respectively to rotate the platform about the external axis and the internal axis.

Therefore, as the first extension and the second extension travel back and forth in the first guide and the second guide respectively the platform rotates about the external axis. However, because the first guide and the second guide are different curved shapes the platform also rotates about the internal axis because the first extension and the second extension will follow different planetary paths. The first extension and second extension can take any form, as can the first guide and second guide, provided the above described movement is facilitated. A more detailed version of this arrangement with further features is described in greater detail below.

The platform is for receiving a person in a prone position, and can comprise an upper surface for the purpose. In terms of the location of the first extension and the second extension relative to the upper surface, they can be level with it on the platform, or they can be above or below it on upper or lower sections or protruding parts of the platform. If they are level with the upper surface, then the first lateral axis and the second lateral axis will be in line with the upper surface. However, if they are displaced either above or below the upper surface on other parts of the platform then the first lateral axis and the second lateral axis will be in planes orthogonal to the upper portion of the platform, which they will share with axes level with the upper surface. To increase the degree of rotation of the upper surface the first lateral axis and the second lateral axis can be different distances from the upper surface. For example, one could be above it and another below it, which would increase the degree of rotation about the internal axis. This would all be a matter for the skilled person according to the specific movements they wanted to create.

In terms of different movements the linkage can facilitate, in alternative embodiments of the present invention the linkage can comprise a linear movement linkage which can support the platform for linear movement relative to the support stand. With this arrangement the user does not experience any swinging or rotation, but linear rocking back and forth. Such versions of the invention would rely on simplex reciprocal linear acceleration to induce relaxation or sleep, which has been shown in the above described studies to be a key factor. The platform can be arranged with the upper portion flat, like a conventional bed, or preferably with the platform at an inclined angle so the user can adopt the Alexander Technique monkey position described above.

In yet another version of the invention the linkage can comprise a rotational linkage which can support the platform for rotation about an internal axis passing through the platform. The purpose of this kind of simplex rotational movement is not to induce relaxation or sleep, but to facilitate ease of use. Namely, as the platform can be rotated on its own internal axis, it can be mounted by the user when it is in a relatively upright position, and then rotated into a comfortable inclined position. Likewise, to make dismounting from the platform easier it can be rotated back into a relatively upright position. The rotation feature removes the possibility of any strain or injury being suffered by the user mounting the platform in an already inclined position.

Preferably some of the above features can be combined to create a multi-function apparatus. In particular, the linkage can comprises a two-phase rotational linkage which can support the platform for rotation about a first internal axis passing through the platform in a first rotation phase, and which can support the platform for rotation about an external axis displaced from the platform in a second rotation phase. The second rotation phase can preferably be a hybrid movement as described above, in which the platform also rotates about an internal axis, which can be the same as the first internal axis, or it can be a different internal axis displaced from the first internal axis (as explained further below).

With this particular version of the invention the support stand can comprise a first portion supporting a first side of the platform, and a second portion supporting a second side of the platform. The platform can comprise a first axle part on the first side thereof, which first axle part can be located for rotation in a first socket provided in the first portion. Likewise the platform can comprise a second axle part on the second side thereof, which second axle part can be located for rotation in a second socket provided in the second portion. The first axle part and the second axle part can be arranged on the first internal axis of the platform about which the platform rotates in the first rotation phase of the two-phase rotational linkage in use.

The first portion and the second portion of the support stand can be any construction capable of supporting the platform for rotation, including A-frames or other upstanding frame structures. However, in one version of the invention the first portion can comprise a first wall, and the second portion can comprise a second wall parallel to the first wall.

The platform can be freely manually rotatable relative to the support stand in the first rotation phase of the two-phase rotational linkage in use. For example, the user can control the angle of rotation by shifting their weight and/or the position of their arms. However, such a freely rotatable platform may be difficult to control and balance safely. Therefore, in one version of the invention the rotational movement of the platform relative to the support stand in the first rotation phase of the two-phase rotational linkage can be limited.

In particular, the platform can then comprise a first boss on the first side, which first boss can be located for movement in a first track provided in the first wall. Likewise the platform can comprise a second boss on the second side, which second boss can be located for movement in a second track provided in the second wall. The first boss and the second boss can be arranged on a second internal axis of the platform, which second internal axis is parallel to the first internal axis and is disposed between the first internal axis and an upper end of the platform. The first track and the second track can comprise shapes which correspond with the planetary path of the second internal axis about the first internal axis during the first rotation phase of the two-phase rotational linkage. With this construction the first track and second track define the rotational movement extent of the platform in the first rotation phase, because the first boss and the second boss will abut against lower ends and upper ends of the first track and second track respectively, and prevent further rotational movement of the platform. This prevents the platform from rotating into an unsafe angle, and it also defines the chosen beneficial angles the platform is capable of achieving in use.

Namely, the support stand can comprise an underside in a second plane, and lower ends of the first track and the second track can delimit rotation of the platform about the first internal axis to a first oblique angle to the second plane. This angle is the furthest the platform will rotate in the first rotation phase, and can be any chosen angle, but is preferably between 60 and 40 degrees to the second plane. The purpose of this is to position the user in the Alexander Technique monkey position in which their torso is angled forward to this kind of extent. The exact angle chosen will be a matter for the skilled person to determine.

In addition, it will be appreciated that upper ends of the first track and the second track also define the maximum rotational angle of the platform at the opposite end of its rotational movement in the first rotation phase. This can be with the first plane substantially normal to the second plane, such that the upper portion is substantially vertical, provided the underside is horizontal. However, preferably the upper ends of the first track and the second track can be positioned such that when the first boss and second boss abut against them the upper portion is at an angle of between 70 and 80 degrees to the second plane. This makes mounting and dismounting the platform an easy and comfortable process for the user.

Moving on from this, the first socket can be located at an inner end of a third track provided in the first wall, and the second socket can be located at an inner end of a fourth track provided in the second wall. The lower end of the first track can be coincident with an inner end of a fifth track provided in the first wall, and the lower end of the second track can be coincident with an inner end of a sixth track provided in the second wall. The third track and fourth track can have a first curved shape, and the fifth track and sixth track can have a second curved shape different to the first curved shape. The first axle part, second axle part, first boss and second boss can be simultaneously moveable through the third track, fourth track, fifth track and sixth track respectively in the second rotation phase of the two-phase rotational linkage in use, in order to rotate the platform about the external axis and a third internal axis passing through the platform. The third internal axis is located between the first internal axis and the second internal axis because the first and second axle parts and first and second bosses follow different curved paths, leading to rotation of the platform about the third axis between them. Its exact location is a factor of the distance between the first internal axis and the second internal axis and the respective shapes of the third and fourth tracks and fifth and sixth tracks.

In order to facilitate all the above described movements the apparatus can comprise a first motorised mechanism for rotating the first axle part and the second axle part in the first socket and the second socket respectively in the first rotation phase of the two-phase rotational linkage in use. The apparatus can then comprise a second motorised mechanism for reciprocally moving the first axle part, second axle part, first boss and second boss along the third track, fourth track, fifth track and sixth track respectively in the second rotation phase of the two-phase rotational linkage in use.

Motorised mechanisms which can achieve the above described movements are well known and are not described herein in any further detail. It will be a matter for the skilled person to determine how best to implement motorised mechanisms to achieve the above results. The first motorised mechanism and the second motorised mechanism can be entirely separate from one another, or they can be part of the same integrated motorised mechanism. The mechanism or mechanisms could be achieved with one or more electric motors supported by power supplies and appropriate circuitry, which electric motors can power transmissions and linkages to control the position and angle of the first axle part, second axle part, first boss and second boss as described above.

In a preferred construction the support stand can comprise a front wall extending between inner ends of the first wall and the second wall, and the support stand can define an internal area between the first wall, front wall and second wall. The first socket and second socket can be located in the first wall and the second wall respectively at a position which is spaced from the front wall such that an upper end of the platform is disposed in the internal area during the first rotation phase of the two-phase rotational linkage in use. Therefore, the support stand can be substantially U-shaped, and can surround the platform. With this construction the platform and user are located in the internal area in use, which provides security and privacy.

The first wall can transition into the front wall in a curved manner, and the second wall can also transition into the front wall in a curved manner. With this construction the support stand has a smooth shape devoid of edges. The first wall, front wall and second wall can all be the same height, but preferably the first wall and second wall can both comprise an inclined upper edge which rises in height from outer ends of the first wall and second wall to the inner ends thereof. The outer ends of the first wall and the second wall can taper inwardly from the upper edges thereof to lower edges thereof. With these shapes the support stand has an attractive and functional shape which is higher at the front to provide greater privacy and security, and lower at the rear to improve user access. In particular the user can support themselves on the upper edges of the first wall or second wall when mounting or dismounting the platform.

The platform can be any shape capable of supporting the user which has an upper portion in a first plane and a lower portion comprising a concavity relative to the first plane. This could be a simplex platform with the upper portion being entirely flat and the lower portion comprising a triangular or square shaped concavity. However, this would not be very comfortable, so preferably the platform can comprise a more ergonomic shape. In particular, the upper portion of the platform which is in the first plane can be a central upper portion of the platform which is aligned with a main axis thereof, and the platform can comprise lateral upper portions which are curved upwardly relative to the central upper portion. With this construction an upper section of the platform made up of the central upper portion and lateral upper portions is concave in a lateral direction thereof, to make it more comfortable to reside on.

Furthermore, the lower portion can comprise a first part inclined relative to the first plane which is shaped and configured to conform in axial length with the thighs of a pre-determined size of user, and a second part which is shaped and configured to confirm in axial length with the knees, shins and ankles of a pre-determined size of user. The upper section can transition into the first part of the lower portion in a curved manner, and the first part can also transition into the second part in a curved manner, so as to make the lower portion more comfortable to reside on.

It will be appreciated that the platform has to be constructed for a pre-determined size of user as it has a static shape, and this can be any chosen size, not only in total height, but also in the relative positions of the head, shoulders, hips, knees and feet. The pre-determined size of user can be an average or mean size of male or female adult or child, but the platform is not restricted to use by people of that specific size. It can still be effectively used by people within a range of sizes and proportions either larger or smaller in size that the pre-determined size. In particular, the apparatus will still function effectively if the user’s hips are merely in the vicinity of the junction between the upper portion and the lower portion as opposed to be directly in line with it. Likewise, if the platform is seven feet in length then anyone shorter than that can utilise it. The pre-determined size used in practice will be a matter for the skilled person to determine.

Like the upper portion, the lower portion can comprise a central lower portion which is aligned with a main axis of the platform, and lateral lower portions which are curved upwardly relative to the central lower portion. With his construction a lower section of the platform made up of the central lower portion and the lateral lower portions is concave in a lateral direction thereof, once again to make it more comfortable for the user.

The platform can be shaped such that the user’s head is proud of the upper portion when their hips are located at the junction of the upper portion and the lower portion. However, preferably the platform can comprise a face aperture for a user’s face to extend through in use. This can be aligned with the main axis of the platform in the area of the central upper portion, and at the point where the head of a user of the pre-determined size would be located when their hips are located at the junction of the upper portion and the lower portion. The face aperture can comprise a shape with a greater length than width in an axial direction of the platform, and rounded ends. With this shape the face aperture can be accessed by users whose heads are located at different points when their hips are located at the junction of the upper portion and the lower portion.

The purpose of the face aperture is two-fold. Firstly, it allows the user to comfortably rest their head in a face-down position, which prevents the need for the head to be twisted to one side, and ensures the neck is kept straight and relaxed. This is an important element of the Alexander Technique monkey position. In this respect the face aperture works like those on known massage or treatment tables. Secondly, the face aperture allows the user to look through the platform, for reasons which are explained below.

In a preferred construction the platform can comprise a pair of arm apertures for a user’s arms to extend through in use. Each arm aperture can be in the area of one of the lateral upper portions, at the points where the shoulders of the user of the pre-determined size would be located when their hips are located at the junction of the upper portion and the lower portion. Again, the arm apertures can each comprise a shape with a greater length than width in an axial direction of the platform, and rounded ends. With these shapes the arm apertures can be accessed by users whose shoulders are located at different points when their hips are located at the junction of the upper portion and the lower portion.

The purpose of the arm apertures is to allow the user’s arms to hang down from their shoulders instead of laying alongside their body, which opens up the muscles of the upper back, relieves strains and tension and improves comfort. It is also an important element of the Alexander Technique monkey position.

The platform can be shaped such that the user’s feet are proud of the lower portion. However, preferably the platform can comprise a pair of feet apertures for a user’s feet to extend through in use. These can be aligned with the second part of the lower portion at points where the feet of the user of the pre-determined size would be located when their hips are located at the junction of the upper portion and the lower portion. Once again, the feet apertures can each comprise a shape with a greater length in an axial direction of the platform, and rounded upper ends. With these shapes the feet apertures can be accessed by users whose feet are located at different points when their hips are located at the junction of the upper portion and the lower portion.

The purpose of the feet apertures is two-fold. Firstly, they allow the user’s feet to protrude through the platform instead of resting on the platform, which prevents the need for the user’s lower legs and/or ankles to be twisted to one side, ensuring that the ankles are kept straight and relaxed. Secondly, the feet apertures make it easier for the user to mount and dismount the platform, and also support some of their weight in any of the plurality of use positions. In particular, the platform can comprise a footrest panel extending from an underside thereof adjacent to lower ends of the pair of feet apertures. As explained in greater detail below, when the platform is in a stand-by position it is arranged generally upright so a user can approach it from a standing position. They can pass their feet through the feet apertures and place them on the footrest panel. From there the user can lean forward onto the platform ready for it to rotate in the first rotation phase of the two-phase rotational linkage. In any of the positions adopted in the first rotation phase and second rotation phase of the two-phase rotational linkage in use the user can support some of their weight through their feet on the footrest panel. The greater the angle achieved in the first rotation phase the less this is required, but it is an important feature to ensure comfort in use, and to prevent the user from sliding down the platform as it rotates and rocks back and forth.

The footrest panel can extend from the underside of the platform at such an angle that it is parallel with the second plane when the first boss and the second boss abut against the upper ends of the first track and the second track respectively. As such, the user can step safely onto and off the footrest platform to mount and dismount the platform.

Reference is made above to the face aperture allowing the user to see through the platform. The purpose of this features is to allow the user to view and/or access a device or devices located in the internal area. Namely, the support stand can comprise one or more user interface devices located inside the internal area. This can be an electronic screen for displaying images for inducing relaxation or sleep and/or audio speakers for playing sounds for the same purpose. They can also be user monitoring devices like biometric monitors and the like as well as temperature and/or air conditioning controls and adjusting functions.

The present invention can be performed in various ways, but five embodiments will now be described by way of example and with reference to the accompanying drawings, in which:

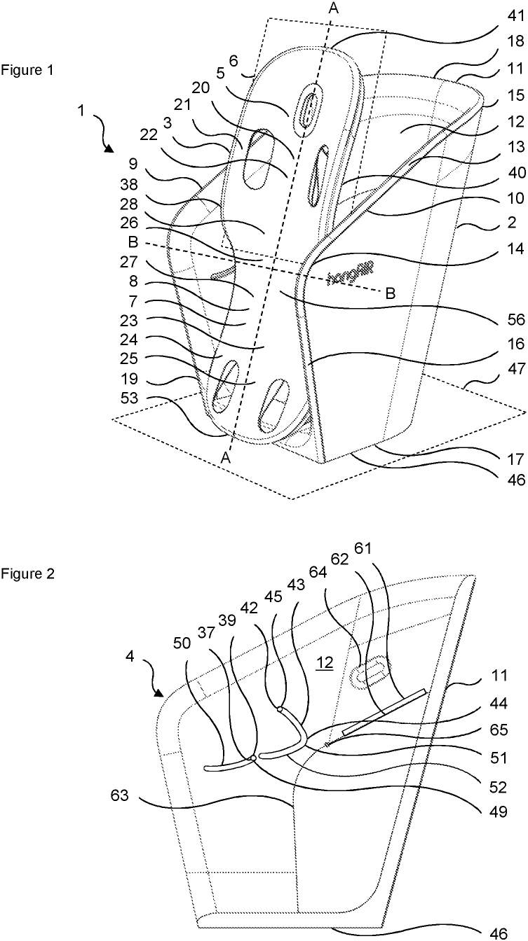

FIG. 1 is a perspective view of a first embodiment of apparatus for supporting a person in a prone position in a stand-by position of a first rotational phase of a two-phase rotational linkage thereof;

FIG. 2 is a cross-sectional side view of the apparatus shown in FIG. 1 with a platform thereof not shown;

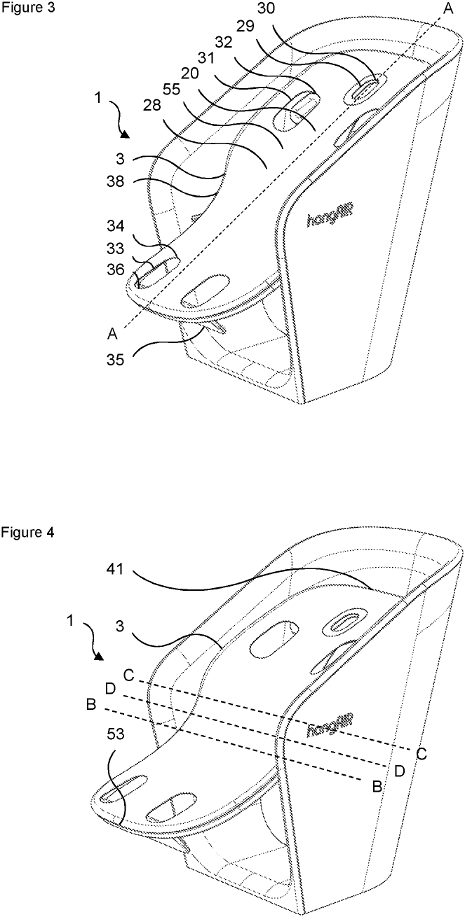

FIG. 3 is a perspective view of the apparatus as shown in FIG. 1 in an inner position of a second rotational phase of the two-phase rotational linkage thereof;

FIG. 4 is a perspective view of the apparatus as shown in FIG. 1 in an outer position of the second rotational phase of the two-phase rotational linkage thereof;

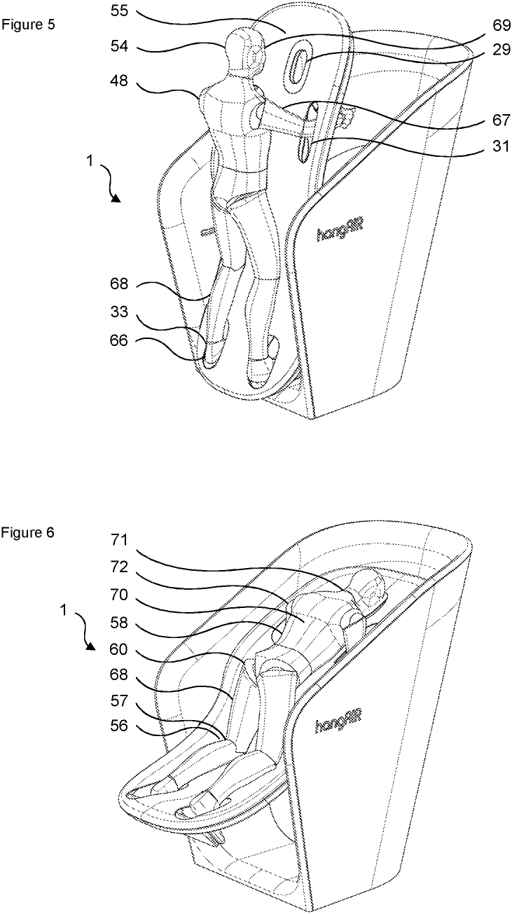

FIG. 5 is a perspective view of the apparatus as shown in FIG. 1 in a stand-by position of the first rotational phase of the two-phase rotational linkage thereof;

FIG. 6 is a perspective view of the apparatus as shown in FIG. 1 in a mid-position of the second rotational phase of the two-phase rotational linkage thereof;

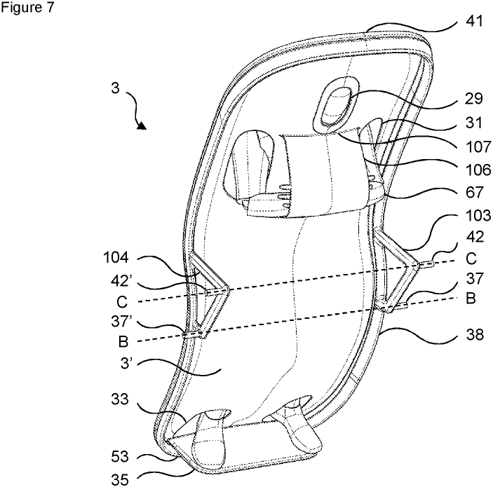

FIG. 7 is a perspective view of a platform of the apparatus shown in FIG. 1 in isolation;

FIG. 8 is a diagrammatic view of a platform and rotational linkage arrangement of a second embodiment of apparatus for supporting a person in a prone position;

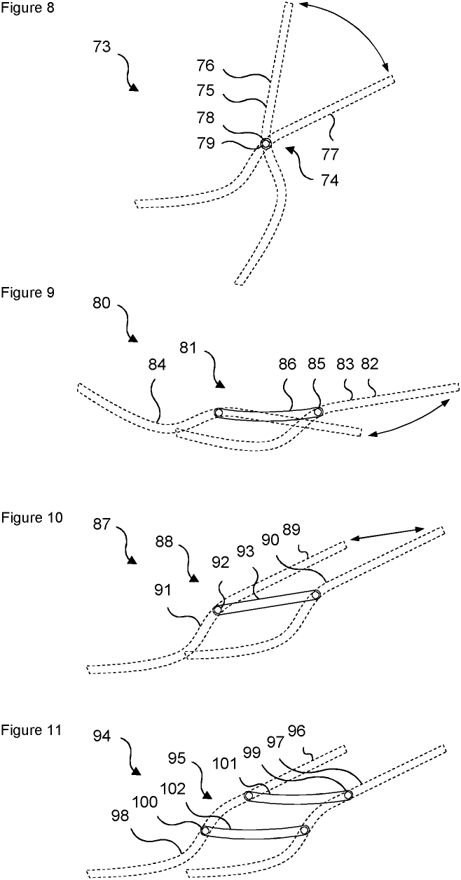

FIG. 9 is a diagrammatic view of a platform and rotational linkage arrangement of a third embodiment of apparatus for supporting a person in a prone position;

FIG. 10 is a diagrammatic view of a platform and rotational linkage arrangement of a fourth embodiment of apparatus for supporting a person in a prone position; and,

FIG. 11 is a diagrammatic view of a platform and rotational linkage arrangement of a fifth embodiment of apparatus for supporting a person in a prone position

As shown in FIG. 1 apparatus for supporting a person in a prone position 1 comprises a support stand 2 and a platform 3 movably mounted to the support stand 2 by a linkage, in the form of two-phase rotational linkage, generally indicated at 4. As explained below, the platform 3 is movable on the linkage 4 between a first position and a second position, which are different in the various different embodiments. The platform 3 comprises an upper portion 5 in a first plane, indicated by hashed box 6, and a lower portion 7 comprising a concavity 8 relative to the first plane 6.

The support stand 2 is a substantially U-shaped part comprising a first wall 9, a second wall 10, and a front wall 11, which collectively define an internal area 12. As is visible from the figures the first wall 9 transitions into the front wall 11 in a curved manner, and the second wall 10 also transitions into the front wall 11 in a similar curved manner. With this construction the support stand 2 has a smooth shape devoid of edges. The first wall 9 and the second wall 10 have a corresponding shape comprising an inclined upper edge 13, which rises in height from an outer end 14 to an inner end 15 thereof, and an outer edge 16 which tapers inwardly from the upper edge 13 to a lower edge 17 thereof. With these shapes of first wall 9 and second wall 10 the support stand 2 has an attractive and functional shape which is higher at the front 18 to provide greater privacy and security, and lower at the rear 19 to improve user access. In particular, the user can support themselves on the upper edges 13 of the first wall 9 or second wall 10 when mounting or dismounting the platform 3.

The platform 3 is moveably mounted to the support stand 2 by the linkage 4, as described in greater detail below. In terms of the shape of the platform 4, it has a generally ergonomic shape suitable for supporting a prone user. Namely the upper portion 5 which is in the first plane 6 is a central upper portion 20 of the platform 3 which is aligned with a main axis A-A thereof. The platform 3 then comprises lateral upper portions 21 which are curved upwardly relative to the central upper portion 20. With this construction an upper section 22 of the platform 3 made up of the central upper portion 20 and lateral upper portions 21 is concave in a lateral direction thereof, to make it more comfortable to reside on.

Like the upper portion 5, the lower portion 7 comprises a central lower portion 23 which is aligned with the main axis A-A of the platform 3, and lateral lower portions 24 which are curved upwardly relative to the central lower portion 23. With this construction a lower section 25 of the platform 3 made up of the central lower portion 23 and the lateral lower portions 24 is concave in a lateral direction thereof, once again to make it more comfortable for the user. Furthermore, the lower portion 7 comprises a first part 26 inclined relative to the first plane 6, which is shaped and configured to conform in axial length with the thighs of a pre-determined size of user, and a second part 27 which is shaped and configured to confirm in axial length with the knees, shins and ankles of a pre-determined size of user. As is visible from the figures the upper section 22 transitions into the lower section 25 in a curved manner, and the first part 26 also transitions into the second part 27 in a curved manner, so as to make the lower section 27 more comfortable to reside on. The lateral upper portions 21 are greater in size than the lateral lower portions 24 and transition thereto accordingly, as shown.

The pre-determined size of user in this case is an average adult size. It will be appreciated that the apparatus 1 can still be effectively used by people within a range of sizes and proportions either larger or smaller in size that the pre-determined size. In particular, the apparatus 1 will still function effectively if the user’s hips are merely in the vicinity of the junction 28 between the upper section 22 and the lower section 25 as opposed to being directly in line with it.

Referring to FIG. 3, the platform 3 comprises a face aperture 29 for a user’s face to extend through in use, which is aligned with the main axis A-A of the platform 3 in the area of the central upper portion 20, and at the point where the head of a user of the pre-determined size would be located when their hips are located at the junction 28 of the upper section 22 and the lower section 25. The face aperture 29 comprise a shape with a greater length than width in an axial direction of the platform 2, and rounded ends 30. With this shape the face aperture 28 can be accessed by users whose heads are located at different points when their hips are located at the junction 28 of the upper section 22 and the lower section 25.

The platform 3 also comprises a pair of arm apertures 31 for a user’s arms to extend through in use. Each arm aperture 31 is in the area of one of the lateral upper portions 21, at the points where the shoulders of the user of the pre-determined size would be located when their hips are located at the junction 28 of the upper section 22 and the lower section 25. Again, the arm apertures 31 each comprise a shape with a greater length than width in an axial direction of the platform 3, and rounded ends 32. With these shapes the arm apertures 31 can be accessed by users whose shoulders are located at different points when their hips are located at the junction 28 of the upper section 22 and the lower section 25.

The platform 3 also comprises a pair of feet apertures 33 for a user’s feet to extend through in use. Each foot aperture 33 is in the area of one of the lateral lower portions 24, in the area of the second part 27 of the lower portion 7, at points where the feet of the user of the pre-determined size would be located when their hips are located at the junction 28 of the upper section 22 and the lower section 25. The feet apertures 33 each comprise a shape with a greater length in an axial direction of the platform 3, and rounded upper ends 34. With these shapes the feet apertures 33 can be accessed by users whose feet are located at different points when their hips are located at the junction 28 of the upper section 22 and the lower section 25.

The platform 3 also comprises a footrest panel 35 extending from an underside 3′ thereof adjacent to lower ends 36 of the pair of feet apertures 33 (most clearly seen in FIG. 7). As explained in greater detail below, when the platform 3 is in a stand-by position as shown in FIG. 1, it is arranged generally upright so a user can approach it from a standing position. They pass their feet through the feet apertures 33 and place them on the footrest panel 35. From there the user can lean forward onto the platform 3 ready for it to rotate in a first rotation phase of the two-phase rotational linkage 4, as described below.

Turning now to the rotational linkage 4, this supports the platform 3 for rotation about a first internal axis B-B passing through the platform 3 in a first rotation phase. It also supports the platform 3 for a hybrid movement during a second rotation phase in which it rotates about an external axis at the same time as rotating independently about a third internal axis D-D.

In order to achieve this the platform 3 comprises a first axle part 37 (which is visible in FIG. 7, and which is indicated by an outline indicated in FIG. 2) on a first side 38 thereof, which first axle part 37 is located for rotation in a first socket 39 provided in the first wall 9. The same arrangement is provided on the second side 40 of the platform 3, where a second axle part 37′ (which is only visible in FIG. 7) is located for rotation in a second socket (not visible) provided in the second wall 10. The first axle part 37 and the second axle part 37′ are arranged on the first internal axis B-B of the platform 3 about which the platform 3 rotates in the first rotation phase of the two-phase rotational linkage 4.

The first socket 39 and second socket are located in the first wall 9 and the second wall 10 respectively at a position which is spaced from the front wall 11 such that an upper end 41 of the platform 3 is disposed in the internal area 12 during the first rotation phase of the two-phase rotational linkage 4 when it rotates about the first internal axis B-B. Therefore, the support stand 2 fully surrounds the platform 3 and provides security and privacy.

The platform 3 also comprises a first boss 42 (which is visible in FIG. 7, and which is indicated by an outline indicated in FIG. 2) on the first side 38, which first boss 42 is located for movement in a first track 43 provided in the first wall 9. The same arrangement is provided on the second side 40 of the platform 3, where a second boss 42′ (which is only visible in FIG. 7) is located for movement in a second track (not visible) provided in the second wall 10. The first boss 42 and the second boss 42′ are arranged on a second internal axis C-C of the platform 3, which second internal axis C-C is parallel to the first internal axis B-B and is disposed between the first internal axis B-B and the upper end of the platform 41.

The first track 43 and the second track comprise curved shapes which correspond with the planetary path of the second internal axis C-C about the first internal axis B-B. With this construction the first track 43 and the second track define the rotational movement extent of the platform 3 in the first rotation phase, because the first boss 42 and the second boss 42′ abut against lower ends 44 and upper ends 45 of the first track 43 and second track respectively, and prevent further rotational movement of the platform 3. This prevents the platform 3 from rotating to an unsafe angle, and it also defines the chosen beneficial angles the platform 3 is capable of achieving in use. Namely, the support stand 2 comprises an underside 46 in a second plane, indicated by hashed box 47, and lower ends 44 of the first track 43 and the second track delimit rotation of the platform 3 about the first internal axis B-B to a first oblique angle to the second plane, as shown in FIG. 3. This angle is the furthest the platform 3 will rotate forwards (in a clockwise direction of the first axle part 37) in the first rotation phase, and in this illustrative example is with the upper portion 5 at 50 degrees to the second plane 47. The purpose of this arrangement is to position the user 48 in the Alexander Technique monkey position in which their torso is tilted forward to this extent, as shown in FIG. 6. (The user 48 is shown in the Figures as a mannequin for illustrative purposes, rather than an actual human.)

In addition, it will be appreciated that the upper ends 45 of the first track 43 and the second track also define the rotational angle of the platform 3 at the opposite end of its rotational movement, as shown in FIGS. 1 and 5, in which it assumes a stand-by position. In this illustrative example this is with the upper portion 5 at an angle of 75 degrees to the second plane 47. In other words, the upper portion 5 is angled a convenient 15 degrees forward from vertical, provided the support stand 2 is placed on a horizontal surface. This makes mounting and dismounting the platform 3 an easy and comfortable process for the user 48. In particular, when the user 48 places their weight on the footrest panel 35 their weight is transmitted though the platform 3 and the first boss 42 and the second boss 42′ to the upper ends 45 of the first track 43 and the second track, because their weight will urge the platform 3 to rotate in an anti-clockwise direction of the first axle part 37. As is clear from FIG. 1 the footrest panel 35 extends from the underside of the platform 3 at 15 degrees, so it is parallel with the second plane 47 in the stand-by position.

Referring back to FIG. 2, the first socket 39 is located at an inner end 49 of a third track 50 provided in the first wall 9. The same arrangement is provided in the second wall 10, where the second socket is located at an inner end of a fourth track (not visible). The lower end 44 of the first track 43 is coincident with an inner end 51 of a fifth track 52 provided in the first wall 9. Once again, the same arrangement is provided in the second wall 10, where the lower end of the second track is coincident with an inner end of a sixth track (not visible) provided in the second wall 10. The third track 50 and fourth track have a first curved shape, and the fifth track 52 and sixth track have a second curved shape different to the first curved shape. In particular, the fifth track 52 and sixth track have a smaller radius than the third track 50 and fourth track.

The first axle part 37, second axle part 37′, first boss 42 and second boss 42′ are simultaneously moveable through the third track 50, fourth track, fifth track 52 and sixth track respectively in the second rotation phase of the two-phase rotational linkage 4, in order to rotate the platform 3 about the external axis and the third internal axis D-D (shown in FIG. 4) passing through the platform 3. The third internal axis D-D is located between the first internal axis B-B and the second internal axis C-C because the first axle part 37 and the second axle part 37′, and the first boss 42 and the second boss 42′ follow different curved paths, leading to rotation of the platform 3 about the third axis D-D between them. In particular, because the radius of the fifth track 52 and the sixth track is smaller than the radius of the third track 50 and the fourth track, the second internal axis C-C rotates more than the first internal axis B-B. All of this means that during rotation of the platform 3 about the external axis, the platform 3 also rotates about the third internal axis D-D such that the upper end 41 of the platform 3 falls and rises further than a lower end 53 of the platform 3. This increases the amount of movement and acceleration experienced by the head 54 of the user 48 during reciprocal movement back and forth, helping to induce relaxation and sleep. The exact location of the third internal axis D-D is a factor of the distance between the first internal axis B- B and the second internal axis C-C, and the respective shapes of the third track 50 and the fourth track, and the fifth track 52 and the sixth track.

The platform 3 comprises an upper surface 55 upon which the user 48 lays down in a prone position, as shown in FIG. 6. The upper surface 55 has the same ergonomic shape as the platform 3 described above, which means it has a depth-wise extent, from the highest part of the lateral upper portions 21 down to a junction 56 between the first part 26 and the second part 27 of the lower portion 7. In terms of the location of the first internal axis B-B relative to the upper surface 55, it passes just below it, as is clear from FIG. 7, because the first axle part 37 and the second axle part 37′ extend laterally from downwardly depending frame extensions 103 and 104 respectively, which extend from the underside 3′ of the platform 3. The first internal axis B-B is still “internal” to the platform 3 as a whole, despite the fact that it does not intersect the upper surface 55. In terms of the location of the second internal axis C-C relative to the upper surface 55, it is also below it because the first boss 42 and the second boss 42′ also extend laterally from the frame extensions 103 and 104 respectively, as shown in FIG. 7. Again, the second internal axis C-C is still “internal” to the platform 3 as a whole. This all means that no part of the user 48 dissects the first internal axis B- B and the second internal axis C-C in use. In effect an axis in the general region of the thighs of the user 48 is located in a plane orthogonal to the first plane 6 which is shares with the first internal axis B-B, and an axis in the general region of the hips 60 of the user 48 is located in a plane orthogonal to the first plane 6 which it shares with the second internal axis C-C.

In order to facilitate all the above described movements the apparatus 1 comprises a first motorised mechanism (not visible) for rotating the first axle part 37 and the second axle part 37′ in the first socket 39 and the second socket respectively in the first rotation phase of the two-phase rotational linkage 4. The apparatus 1 also comprises a second motorised mechanism (not visible) for reciprocally moving the first axle part 37, second axle part 37′, first boss 42 and second boss 42′ along the third track 50, fourth track, fifth track 52 and sixth track respectively in the second rotation phase of the two-phase rotational linkage 4. Such motorised mechanisms are known and are not described herein in any further detail.

In addition, the apparatus 1 is provided with a PCB control device (not visible) for activating the first motorised mechanism and second motorised mechanism accordingly. In particular, the PCB control device is provided with programming which determines the speed and acceleration of the platform 3 in both the first rotation phase and the second rotation phase of the two-phase rotational linkage 4. In the first rotation phase the rotation of the platform 3 about the first internal axis B-B is relatively slow in order to maintain safety, namely a speed of 5 cm per second at the upper end 41 of the platform 3. In the second rotation phase one full oscillation of the platform 3 back and forth takes 4 seconds, at a speed of 15 cm per second. To achieve this the second motorised mechanism accelerates the platform 3 from its inner and outer positions shown in FIGS. 3 and 4 respectively, up to a mid-position as shown in FIG. 6 at 0.3 meters per second squared, and then decelerates it from the mid-point to the inner and outer positions at -0.3 meters per second squared.

The support stand 2 comprises an electronic screen 61 on the top surface 62 of a pedestal 63 located in the internal area 12. It also comprises a pair of audio speakers (only one of which 64 is visible) on the first wall 9 and the second wall 10. The electronic screen 61 and the audio speakers 64 are controlled by the PCB control device, and in use the screen 61 shows images intended to induce relaxation and sleep, and the audio speakers 64 emit sounds intended to induce relaxation and sleep. Such images and sounds are known and are not described herein in any further detail. The support stand 2 also comprises a biometric monitor 65 on the pedestal 63 for monitoring the biometrics of the user 48.

Referring to FIG. 7, the underside 3′ of the platform 3 comprises an arm sling 105, located between the arm apertures 31. The arm sling 105 comprises a rectangular band of flexible material attached at both ends (only one of which 106 is visible) to the underside 3′ of the platform 3 at positions closer together than the length of the band, so the sling is formed. The user 48 can slot their arms 67 through the arm sling 105 in the manner shown in FIG. 7 to increase comfort, and to take some of the load of the weight of their arms 67 off their shoulders 72.

The apparatus 1 also comprises a control panel (not visible) via which the user 48 or a supervisor can operate the apparatus 1 in the manners described above. The control panel allows choices to be made about the kinds and durations of movements to be performed, for example 30 minutes of reciprocal actions of the second rotation phase of the two-phase rotational linkage 4, and/or a choice of images and sounds to be emitted. Once again, control panels which can facilitate this kind of electronic control are known, so the control panel is not described herein in any further detail.

In use the apparatus 1 is initially arranged in the stand-by position as shown in FIGS. 1 and 5. In this position the first boss 42 and the second boss 42′ are located at the upper ends 45 of the first track 43 and the second track respectively, and subsequently the upper portion 5 of the platform 3 is arranged at 75 degrees to the second plane 47. The platform 3 is moved to this position, and is maintained in this position, by the first motorised mechanism acting on the first axle part 37 and the second axle part 37′.

A movement programme is entered into the control panel, either by the user 48 or a supervisor. Choices are made about the duration of operation and the choices of images and sounds to be emitted. The PCB control device delays operating the apparatus 1 for a pre-determined period of time to allow the user 48 to mount the platform 3.

To mount the platform 3 the user 48 passes their feet 66 through the feet apertures 33 and places them on the footrest panel 35, and then passes their arms 67 through the arm apertures 31, as shown in FIG. 5. In stepping onto the platform 3 in this way the user 48 can use the upper edges 13 of the first wall 9 and the second wall 10 to steady themselves. The user 48 then leans forward onto the upper surface 55, and in doing so they adopt a squatting posture with their legs 68 bent into the lower portion 7, with their knees 57 placed against the junction 56 between the first part 26 and the second part 27 of the lower portion 7. They also place their face 69 through the face aperture 29. In this position the user 48 will have to support some of their weight through their legs 68.

The first motorised mechanism then rotates the first axle part 37 in a clockwise direction and the second axle part 37′ in an anti-clockwise direction at a speed of 5 cm per second at the upper end 41 of the platform 3. This rotates the platform 3 about the first internal axis B-B and constitutes the first rotation phase of the two-phase rotational linkage 4. The rotation speed is slow enough for the user 48 to remain safely in place on the platform 3, and continues until the first boss 42 and the second boss 42′ reach the lower ends 44 of the first track 43 and the second track. This occurs when the platform 3 reaches an angle of 50 degrees to the second plane 47, as shown in FIG. 3. Once the platform 3 is in this position the user 48 is placed in the Alexander Technique monkey position described above, as shown in FIG. 6. Namely, the user 48 is in a face-down prone position in which their back 70 and neck 71 remain in a neutral aligned position with their head 54 on the upper portion 5, while their hips 60 are in a relaxed rotated pelvic position relative to their back 70. Their knees 57 are also in a relaxed bent position in the lower portion 7, and their arms 67 extend down in a relaxed forward position from their shoulders 72. As mentioned above the user 48 can place their arms 67 in the arm sling 105 as shown in FIG. 7, in order to increase comfort and to take some of the load of the weight of their arms 67 off their shoulders 72. Further, as their feet 66 are located in the feet apertures 33 their legs 68 are in a relaxed forward-facing position without any twisting of the knees 57 or hips 60. As explained above the placement of the user 48 in this particular position mitigates strains and stresses which would otherwise be placed on the user 48 if they adopted other sitting or lying positions. It should also help to address any aches and pains the user 48 is experiencing, by opening the back out, straightening the neck, allowing the hips and knees to bend and integrating the limbs with the core of the body.

Once the platform 3 has achieved the angled forward position shown in FIG. 3, it is in the inner position of the second rotation phase of the two-phase rotational linkage 4, and the second rotation phase can then begin. In particular, the first boss 42 and the second boss 42′ are now at the inner ends 49 of the fifth track 52 and the sixth track respectively, and are able to move along them. The second motorised mechanism then reciprocally moves the first axle part 37, second axle part 37′, first boss 42 and second boss 42′ along the third track 50, fourth track, fifth track 52 and sixth track respectively. One full oscillation of the platform 3 back and forth takes 4 seconds, at an average speed of 15 cm per second. To achieve this the second motorised mechanism accelerates the platform 3 from its inner and outer positions shown in FIGS. 3 and 4 respectively, up to a mid-position as shown in FIG. 6 at 0.3 meters per second squared, and then decelerates it from the mid-point to the inner and outer positions at -0.3 meters per second squared. It is understood that this gentle rocking motion will induce relaxation and sleep because the vestibular otolithic organs mediate sleep promotion, and this effect is a result of repeated acceleration back and forth experienced by the user 48.

During these reciprocal movements the platform 3 is rotated about the external axis as well as the third internal axis D-D, for the reasons explained above. What is important is that this hybrid movement results in the upper end 41 of the platform 3 falling and rising further than the lower end 53. This increases the amount of movement and acceleration experienced by the head 54 of the user 48 during the reciprocal movement back and forth, helping to induce relaxation and sleep.

During the second rotation phase of the two-phase rotational linkage the electronic screen 61 displays the chosen images and the audio speakers 64 emit the chosen sounds. As explained above, these are designed to induce relaxation and sleep. The user 48 views the electronic screen 61 through the face aperture 29. In addition, the biometric monitor 65 monitors the user 48 and collects biometric data which is stored in the PCB control device for further analysis.

During the second rotation phase of the two-phase rotational linkage 4 the upper portion 5 and the first part 26 of the lower portion 7 are located in the internal area 12, behind the front wall 11 and between the first wall 9 and the second wall 10. As such, the user 48 is located in a safe and secure space.

Once the chosen programme of movements has been completed, for example after 30 minutes, the second motorised mechanism brings the platform 3 to a halt in the inner position shown in FIG. 3. In this position the first axle part 37 and the second axle 37′ part are back in the first socket 39 and the second socket respectively, and the first boss 42 and the second boss 42′ are at the lower ends 44 of the first track 43 and the second track, and can move up them once again. The first motorised mechanism then rotates the first axle part 37 in an anti-clockwise direction and the second axle part 37′ in a clockwise direction in a reverse of the above described movement. This is continued until the first boss 42 and the second boss 42′ are located at the upper end 45 of the first track 43 and the second track, and the platform 3 is back in the stand-by position shown in FIGS. 1 and 5. The user 48 can then dismount the platform 3 in a reverse of the above described action.

The present invention can be altered without departing from the scope of claim 1. For example, in a second apparatus 73 illustrated diagrammatically in FIG. 7, the linkage 74 is just a rotational linkage which supports the platform, shown by hashed outline 75, for rotation about an internal axis passing therethrough. FIG. 7 shows the first position 76 and the second position 77 of the platform 75, which are achieved by means of axle part 78, which is aligned with the internal axis, rotating in socket 79. The purpose of this kind of simplex rotational movement is not to induce relaxation or sleep, but simply to facilitate ease of use. Namely, as the platform 75 can be rotated on its own internal axis it can be mounted by the user when it is in its relatively upright first position 76, and then rotated into its comfortable inclined second position 77, where it can remain for a period of relaxation time. Likewise, to make dismounting from the platform 74 easier it can be rotated back into the relatively upright first position 76.

In third apparatus 80 illustrated diagrammatically in FIG. 8, the linkage is a rotational linkage 81 which supports the platform, shown by hashed outline 82, for rotation about an external axis displaced from the platform 82. FIG. 8 shows the first position 83 and the second position 84 of the platform 82, which are achieved by boss 85 moving reciprocally back and forth in curved track 86. This is done without the angle of the platform 82 changing relative to the external axis, which means that it simply swings back and forth. The purpose of this kind of simplex rotational movement is simply to rock the user back and forth in this way to induce relaxation or sleep. The duration of one oscillation, and the acceleration and deceleration rates can be the same as in apparatus 1 described above, or can be made to be different.

In fourth apparatus 87 illustrated diagrammatically in FIG. 9, the linkage is a linear linkage 88 which supports the platform, shown by hashed outline 89, for simplex linear reciprocal movement. FIG. 9 shows the first position 90 and the second position 91 of the platform 89, which are achieved by boss 92 moving reciprocally back and forth in linear track 93. This is done without the angle of the platform 89 changing from that shown, which is a comfortable forward inclined position suitable to achieve the Alexander Technique monkey position as described above. The purpose of this simplex linear movement is simply to rock the user back and forth in a linear fashion rather than in any swinging or rotating manner. Again, the duration of one oscillation, and the acceleration and deceleration rates can be the same as in apparatus 1 described above, or can be made to be different.

In fifth apparatus 94 illustrated diagrammatically in FIG. 10, the linkage is a parallel rotational linkage 95 which supports the platform, shown by hashed lines 96, for rotation about an external axis displaced from the platform 96. FIG. 10 shows the first position 97 and the second position 98 of the platform 96, which are achieved by first boss 99 and second boss 100 moving reciprocally back and forth in first curved track 101 and second curved track 102. The first curved track 101 and the second curved track 102 are identical, which means that the angle of the platform 96 remains the same throughout its motion, and it does not rotate on any internal axis thereof. In this configuration an internal axis of the platform 96 which is equidistant between the first boss 99 and the second boss 100 is effectively that which defines the shape of the arc along which the platform 96 travels, and which rotates about the external axis. The purpose of this rotational movement is to rock the user back and forth to induce relaxation and sleep without rotating the user on their own axis. Once again, the duration of one oscillation, and the acceleration and deceleration rates can be the same as in apparatus 1 described above, or can be made to be different.

The apparatus of the invention can be altered in other ways without departing from the scope of claim 1. For example, in one alternative embodiment (not shown) the platform is substantially flat in its lateral extent, and does not feature any lateral upper portions or lateral lower portions.

In other alternative embodiments (not shown), the PCB control device can move the platform back and forth both faster and slower than as described above. This may be preferred if further research indicates that different speeds and/or rates of acceleration to those described above are advantageous.

Therefore, the apparatus of the present invention provides a platform for supporting a user in a face-down prone position in which their back and neck can remain in a neutral aligned position on the upper portion, while their hips can assume a relaxed rotated pelvic position relative to their spine and their knees can assume a relaxed bent position in the lower portion. This is essentially the beneficial Alexander Technique monkey position. The linkage between the support stand and the platform can then provide a variety of different advantageous movements to induce relaxation or sleep and/or to facilitate ease of use. In particular, the two-phase rotational linkage can do both, and in the second rotation phase can provide a particularly beneficial hybrid rotational movement in which the platform rotates about an external axis as well as an internal axis.

1. Apparatus for supporting a person in a prone position comprises a support stand and a platform movably mounted to the support stand by a linkage, in which the platform is movable on said linkage between a first position and a second position, in which the platform comprises an upper portion in a first plane and a lower portion comprising a concavity relative to the first plane.

2. Apparatus for supporting a person in a prone position as claimed in claim 1 in which the linkage comprises a rotational linkage which supports the platform for rotation about an external axis displaced from the platform.

3. Apparatus for supporting a person in a prone position as claimed in claim 2 in which the rotational linkage supports the platform at an angle to the support stand in the first position, and in which the rotational linkage supports the platform such that it remains at the angle between the first position and the second position.

4. Apparatus for supporting a person in a prone position as claimed in claim 2 in which the rotational linkage also supports the platform for independent rotation about an internal axis passing through the platform during rotation of the platform about the external axis, in which the support stand comprises an underside in a second plane, in which the rotational linkage supports the platform at a first oblique angle to second plane in the first position, and at a second oblique angle to second plane in the second position.

5. Apparatus for supporting a person in a prone position as claimed in claim 4 in which the rotational linkage comprises a first extension on a first side of the platform, which first extension located in a first guide provided in the support stand, and a second extension on the first side of the platform, which second extension is located in a second guide provided in the support stand, in which the first extension is located on a first lateral axis of the platform, in which the second extension is located on a second lateral axis of the platform which is parallel to the first lateral axis and is disposed between the first lateral axis and an upper end of the platform, in which the first guide has a first curved shape, in which the second guide has a second curved shape different to the first curved shape, and in which the first extension and the second extension are simultaneously moveable through the first guide and the second guide respectively to rotate the platform about the external axis andthe internal axis.

6. Apparatus for supporting a person in a prone position as claimed in claim 1 in which the linkage comprises a linear movement linkage which supports the platform for linear movement relative to the support stand.

7. Apparatus for supporting a person in a prone position as claimed in claim 1 in which the linkage comprises a rotational linkage which supports the platform for rotation about an internal axis passing through the platform.

8. Apparatus for supporting a person in a prone position as claimed in claim 1, in which the linkage comprises a two-phase rotational linkage which supports the platform for rotation about a first internal axis passing through the platform in a first rotation phase, and which supports the platform for rotation about an external axis displaced from the platform in a second rotation phase.

9. Apparatus for supporting a person in a prone position as claimed in claim 8 in which the support stand comprises a first portion supporting a first side of the platform, and a second portion supporting a second side of the platform, in which the platform comprises a first axle part on the first side thereof, which first axle part is located for rotation in a first socket provided in the first portion, and in which the platform comprises a second axle part on the second side thereof, which second axle part is located for rotation in a second socket provided in the second portion, and in which the first axle part and the second axle part are arranged on the first internal axis of the platform about which the platform rotates in the first rotation phase of the two-phase rotational linkage in use.

10. Apparatus for supporting a person in a prone position as claimed in claim 9 in which the first portion comprises a first wall, in which the second portion comprises a second wall parallel to the first wall, in which the platform comprises a first boss on the first side, which first boss is located for movement in a first track provided in the first wall, in which the platform comprises a second boss on the second side, which second boss is located for movement in a second track provided in the second wall, in which the first boss and the second boss are arranged on a second internal axis of the platform, which second internal axis is parallel to the first internal axis and is disposed between the first internal axis and an upper end of the platform, and in which the first track and the second track comprise shapes which correspond with the planetary path of the second internal axis about the first internal axis.

11. Apparatus for supporting a person in a prone position as claimed in claim 10 in which the support stand comprises an underside in a second plane, and in which lower ends of the first track and the second track delimit rotation of the platform about the first internal axis to a first oblique angle to the second plane.

12. Apparatus for supporting a person in a prone position as claimed in claim 11 in which the first socket is located at an inner end of a third track provided in the first wall, in which the second socket is located at an inner end of a fourth track provided in the second wall, in which the lower end of the first track is coincident with an inner end of a fifth track provided in the first wall, in which the lower end of the second track is coincident with an inner end of a sixth track provided in the second wall, in which the third track and fourth track have a first curved shape, in which the fifth track and sixth track have a second curved shape different to the first curved shape, and in which the first axle part, second axle part, first boss and second boss are simultaneously moveable through the third track, fourth track, fifth track and sixth track respectively to rotate the platform about the external axis and a third lateral axis passing through the platform in the second rotation phase of the rotational linkage in use.

13. Apparatus for supporting a person in a prone position as claimed in claim 12 in which the apparatus comprises a first motorised mechanism for rotating the first axle part and the second axle part in the first socket and the second socket respectively in the first rotation phase of the two-phase rotational linkage in use, and in which the apparatus comprises a second motorised mechanism for reciprocally moving the first axle part, second axle part, first boss and second boss along the third track, fourth track, fifth track and sixth track respectively in the second rotation phase of the two-phase rotational linkage in use.

14. Apparatus for supporting a person in a prone position as claimed in claim 10 in which the support stand comprises a front wall extending between inner ends of the first wall and the second wall, in which the support stand defines an internal area between the first wall, front wall and second wall, and in which the first socket and the second socket are located in the first wall and the second wall at a position which is spaced from the front wall such that an upper end of the platform is disposed in the internal area during the first rotation phase of the two-phase rotational linkage in use.

15. Apparatus for supporting a person in a prone position as claimed in claim 1 in which the platform comprises a face aperture for a user’s face to extend through in use.

16. Apparatus for supporting a person in a prone position as claimed in claim 1 in which the platform comprises a pair of arm apertures for a user’s arms to extend through in use.

17. Apparatus for supporting a person in a prone position as claimed in claim 1 in which the platform comprises a pair of feet apertures for a user’s feet to extend through in use.

18. Apparatus for supporting a person in a prone position as claimed in claim 17 in which the platform comprises a footrest panel extending from an underside thereof adjacent to lower ends of the pair of feet apertures.
